(19) United States
(12) Patent Application Publication
(10) Pub. No.: US 2005/0249789 A1
(43) Pub. Date: Nov. 10, 2005

(54) Alpha/beta hydrolase-fold enzymes

(76) Inventor: David A. Estell, San Mateo, CA (US)

Correspondence Address: GENENCOR INTERNATIONAL, INC.;ATTENTION: LEGAL DEPARTMENT, 925 PAGE MILL ROAD, PALO ALTO, CA, 94304, US

(21) Appl. No.: 11/179,977

(22) Filed: Jul. 12, 2005

Related U.S. Application Data
(63) Continuation of application No. 09/950,368, filed on Sep. 10, 2001.

(30) Foreign Application Priority Data
Nov. 19, 1998 (WO) PCT/US98/24973
Nov. 20, 1997 (GB) 9724629.2

(51) Int. Cl. C07H 21/04; C12N 9/54; C12P 21/06; C11D 3/386; A23K 1/165; C12N 15/74

(52) U.S. Cl. 424/442; 510/320; 435/69.1; 435/221; 435/252.31; 435/471; 536/23.2

(57) ABSTRACT

The present invention relates to the identification of novel hydrolases in gram positive microorganisms. The present invention provides amino acid sequences for the hydrolase. The present invention provides host cells which comprise nucleic acid encoding the hydrolase. The present invention also provides cleaning compositions, animal feeds and compositions used to treat a textile that include the hydrolase of the present invention.

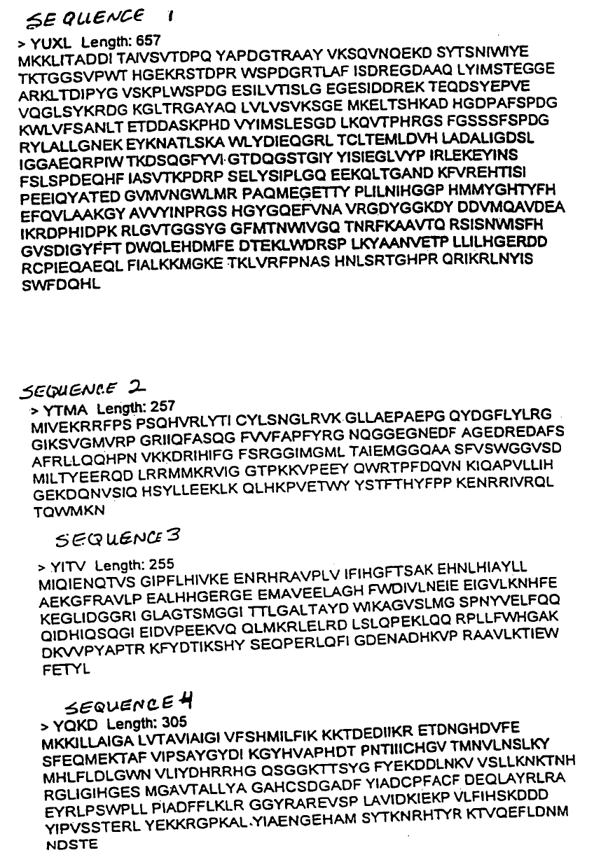

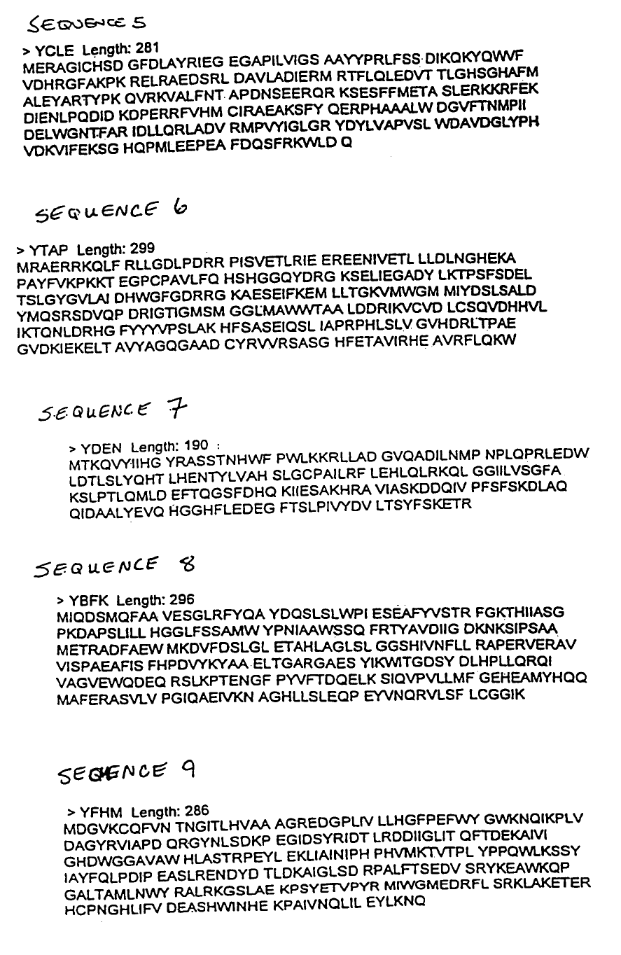

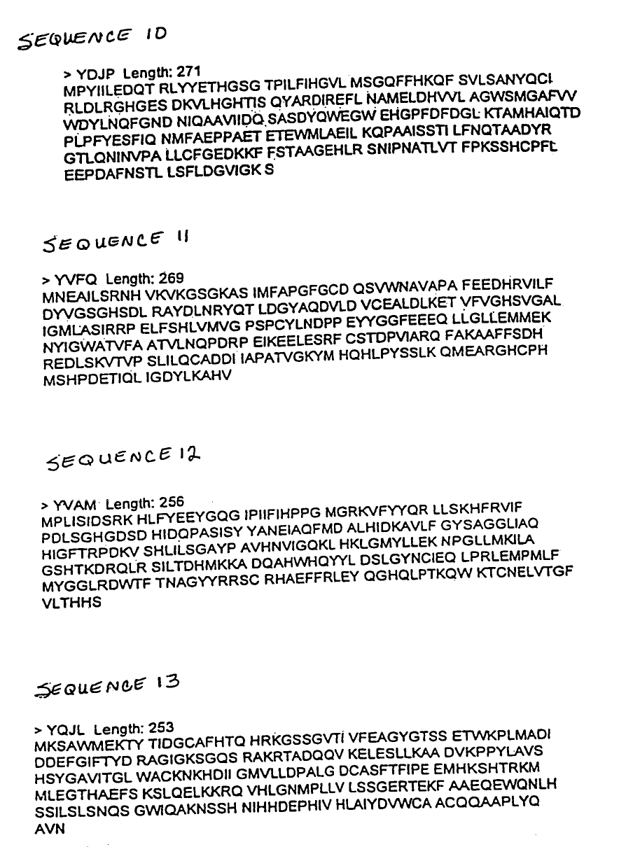

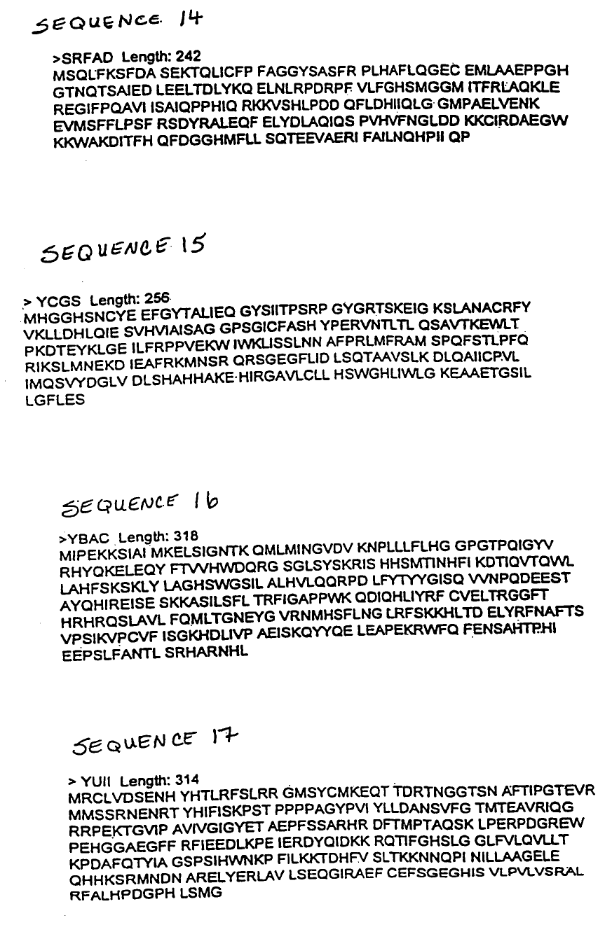

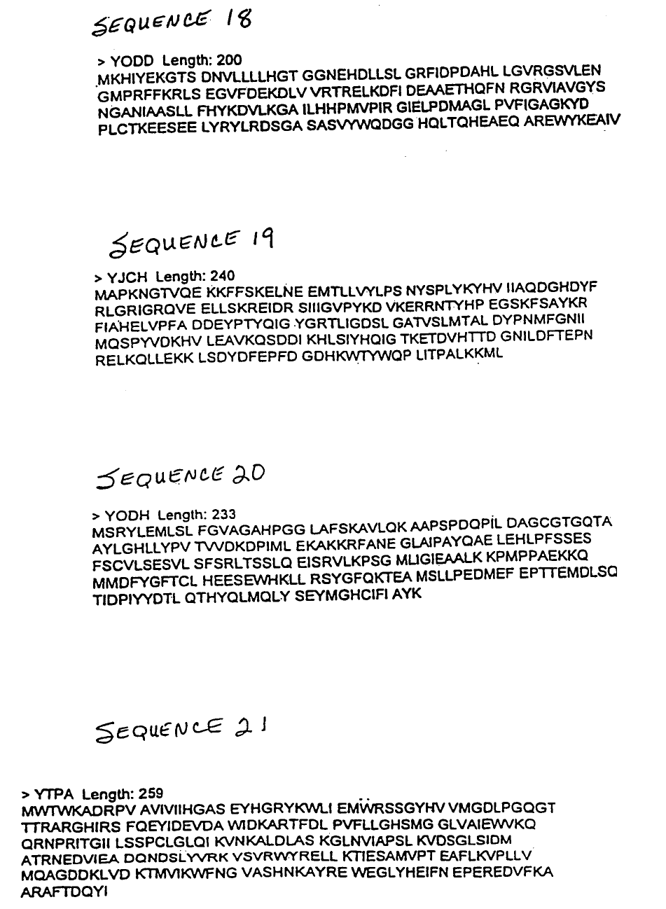

### FIELD OF THE INVENTION

The present invention relates to alpha/beta hydrolase-fold enzymes derived from gram-positive microorganisms. The present invention provides the nucleic acid and amino acid sequences for the hydrolases and methods for their use.

### BACKGROUND OF THE INVENTION

The alpha/beta hydrolase fold common to several hydrolytic enzymes of differing phylogenetic origin and catalytic function was described by Ollis et al. (1992, Protein Eng. 5(3): 197-211). The core of each enzyme in this family was described as being similar: an alpha/beta sheet of eight beta-sheets connected by alpha-helices with conserved arrangement of catalytic residues. Members of this family were found to have a catalytic triad which is borne on the conserved loop structure found in the fold. In the five members discussed in Ollis et al., the is catalytic residues always occur in the same order in the primary sequence: nucleophile, acid, histidine. Furthermore, the catalytic triad residues of the members had similar topological and three dimensional positions.

Members of the hydrolase family include a hydroxylyase (Wajant, et al., 1996, J. Biol. Chem. 271(42):25830-25834) which comprises the active site motif Gly-X-Ser-X-Gly/Ala and the residues Serine 80, Aspartic 208, and Histidine 236 which are critical for enzyme activity; 2-hydroxymuconic semialdehyde hydrolase, XylF (Diaz E., 1995, J. Biol. Chem. 270 (11):6403-6411), which comprises the residues Ser107, Asp228 and His 256; non-heme haloperoxidases ( ) comprises oxidases, which perform halogenation and which are related to esterases (Pelletier et al., 1995, Biochim Biophys Acta, 1995, 1250(2):149-157) which comprises the residues Serine 97, Aspartic acid 229, Histidine 258; and dipeptidyl-peptidase IV (David et al., 1993, J. Biol. Chem. 268(23):17247-17252), which comprises the residues Ser624Asp702, His734).

### SUMMARY OF THE INVENTION

The present invention relates to the identification of gram-positive microorganism members of the family of alpha/beta hydrolase fold enzymes which are characterized by structural relatedness and which comprises conserved catalytic triads. These newly identified members of this family can be used in industrial applications, e.g., the textile industry, in cleaning compositions, such as detergents, and in animal feeds.

Accordingly, the present invention provides compositions comprising a hydrolase selected from the group consisting of YUXL, YTMA, YITV, YQKD, YCLE, YTAP, YDEN, YBFK, YFHM, YDJP, YVFQ, YVAM, YQJL, SRFAD, YCGS, YTPA, YBAC, YUII, YODD, YJCH, YODH which can be used in detergent compositions, compositions for the treatment of textiles; and animal feeds, for example. The present invention also provides commercial applications of the compositions, e.g., their use in methods for treating textiles and methods for cleaning.

The present invention provides amino acid sequences for hydrolases obtainable from B. subtilis (B. subtilis hydrolases). Due to the degeneracy of the genetic code, the present invention encompasses any nucleic acid sequence that encodes the specific hydrolase amino acid sequence shown in the Sequences.

The present invention provides methods for detecting gram positive microorganism homologs of the B. subtilis hydrolases that comprise hybridizing part or all of the nucleic acid encoding the hydrolase with nucleic acid derived from gram-positive organisms, either of genomic or cDNA origin. In one embodiment, the gram-positive microorganism includes B. lichenifonuis, B. lentus, B. brevis, B. stearothermophilus, B. alkatophilus, B. amyloliquefadens, B. coagulans, B. circulans, B. lautus and Bacillus thuringiensis.

The invention further provides for a hydrolase that has at least 80%, at least 85%, at least 90%, and at least 95% homology with the specific amino acid sequences shown in the sequences. The invention also provides for a nucleic acid which encodes a hydrolase that has at least 80%, at least 85%, at least 90% and at least 95% homology with the naturally occurring nucleic acid sequence found in Bacillus subtilis.

In a preferred embodiment, the present invention provides the naturally occurring hydrolase nucleic acid molecule having the sequence found in Bacillus subtilis 1-168 strain (Bacillus Genetic Stock Center, accession number 1A1, Columbus, Ohio) as disclosed infra.

The present invention provides expression vectors and host cells comprising a nucleic acid encoding a gram positive hydrolase. The present invention also provides methods of making the gram positive hydrolase.

The present invention encompasses novel amino acid variations of hydrolase amino acid sequences from gram positive microorganisms disclosed herein that have hydrolytic activity. Naturally occurring hydrolases derived from gram positive microorganisms disclosed herein as well as proteolytically active amino acid variations or derivatives thereof, have application in the textile industry, in cleaning compositions and methods and in animal feed compositions.

In another embodiment, a host cell is engineered to produce a hydrolase of the present invention. In a further aspect of the present invention, a hydrolase from a gram positive microorganism is produced on an industrial fermentation scale in a host expression system. The host cell may be a gram-negative or gram-positive microorganism, a fungal organism, or higher Eucaryotes. Gram negative microorganisms include but are not limited to members of Enterobacteriaceae; gram positive microorganisms include but are not limited to members of Bacillus and Pseudomonase; fungal organisms include but are not limited to Aspergillus and Tricoderna; and higher eucaryotes include mammalian cells.

The gram positive microorganism host cell may be normally sporulating or non-sporulating and may be modified in other ways to facilitate expression of the hydrolase. In a preferred embodiment, the gram positive host cell is a Bacillus. In another embodiment, the Bacillus includes B. subtilis, B. licheniformis, B. lentus, B. brevzs B. stearothernophilus, B. alkalophilus, B. amyloliquefaciens, B. coagulans, B. circulans, B. lautus and B. thuringiensis. In a further preferred embodiment, the Bacillus host cell is Bacillus subtilis.

### DETAILED DESCRIPTION

Definitions

The present invention relates to a newly characterized hydrolases from gram positive organisms. In a preferred embodiment, the gram positive organisms is a Bacillus. In another is preferred embodiment, the Bacillus includes B. subtilis, B. licheniformis, B. lentus, B. brevis, B. stearothermophilus, B. alkalophilus, B. amyloliquefaciens, B. coagulans, B. circulans, B. lautus and B. thuringiensis.

In another preferred embodiment, the gram positive organism is Bacillus subtilis and the hydrolases have the amino acid sequence disclosed in the Sequences. In a preferred embodiment, the hydrolase is encoded by the naturally occurring nucleic acid that is found at the respective positions of B. subtilis detailed infra.

As used herein, “nucleic acid” refers to a nucleotide or polynucleotide sequence, and fragments or portions thereof, and to DNA or RNA of genomic or synthetic origin which may be double-stranded or single-stranded, whether representing the sense or antisense strand. As used herein “amino acid” refers to peptide or protein sequences or portions thereof. A “polynucleotide homologue” as used herein refers to a gram positive microorganism polynucleotide that has at least 80%, at least 85%, at least 90% and more preferably at least 95% identity to B. subtilis hydrolase, or which is capable of hybridizing to B. subtilis hydrolase under conditions of high stringency and which encodes an amino acid sequence having hydrolase activity.

The terms “isolated” or “purified” as used herein refer to a nucleic acid or peptide or protein that is removed from at least one component with which it is naturally associated. As used herein, isolated nucleic acid can include a vector comprising the nucleic acid.

As used herein, the term “overexpressing” when referring to the production of a protein in a host cell means that the protein is produced in greater amounts than in its naturally occurring environment.

As used herein, the phrase “hydrolytic activity” refers to a protein that is able to hydrolyze a peptide bond. Enzymes having proteolytic activity are described in Enzyme Nomenclature, 1992, edited Webb Academic Press, Inc.

I. Hydrolase Sequences

The present invention encompasses the use of hydrolase polynucleotide homologues encoding gram positive microorganism hydrolases which have at least 80%, at least 85%, at least 90%, and at least 95% identity to naturally occurring B. subtilis hydrolase nucleic acid as long as the homologue encodes a protein that has hydrolytic activity.

Gram positive polynucleotide homologues of B. subtilis hydrolase may be obtained by standard procedures known in the art from, for example, cloned DNA (e.g., a DNA “library”), genomic DNA libraries, by chemical synthesis once identified, by cDNA cloning, or by the cloning of genomic DNA, or fragments thereof, purified from a desired cell. (See, for example, Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.) A preferred source is from genomic DNA.

As will be understood by those of skill in the art, the amino acid sequence disclosed in the Sequences may reflect inadvertent errors inherent to nucleic acid sequencing technology. The present invention encompasses the naturally occurring nucleic acid molecule having the nucleic acid sequence obtained from the genomic sequence of Bacillus species and the naturally occurring amino acid sequence.

Nucleic acid encoding Bacillus subtilis hydrolase starts around the kilobases shown in Table I counting from the point of origin in the Bacillus subtilis strain 1-168 (Anagnostopala, 1961, J. Bacteriol., 81:741-746 or Bacillus Genomic Stock Center, accession 1A1, Columbus, Ohio). The Bacillus subtilis point of origin has been described in Ogasawara, N. (1995, Microbiology 141:Pt.2 257-59). Bacillus subtilis hydrolase has a length of 415 amino acids. Based upon the location of the DNA encoding Bacillus subtilis hydrolase, naturally occurring B. subtilis hydrolase can be obtained by methods known to those of skill in the art including PCR technology. The nucleotide sequence for the hydrolases disclosed in Table I can be found in Nature 1997 vol 390, pages 249-256.

TABLE I

| hydrolase | kb from |
|---|---|
| designation | pt of origin |
| YUXL | 3111 |
| YTMA | 3131 |
| YITV | 1190 |
| YQKD | 2459 |
| YCLE | 414 |
| YTAP | 3095 |
| YDEN | 573 |
| YDJP | 682 |
| YVFQ | 3496 |
| YQJL | 2476 |
| SRFAD | 401 |
| YCGS | 352 |
| YTPA | 3122 |
| YBAC | 134 |
| YUII | 3291 |
| YODD | 2128 |
| YODH | 2132 |
| YBFK | 246 |
| YVAM | 3452 |
| YFHM | 929 |

Oligonucleotide sequences or primers of about 10-30 nucleotides in length can be designed from the naturally occurring polynucleotide sequences and used in PCR technology to isolate the naturally occurring sequence from B. subtilis genomic sequences.

Another general strategy for the “cloning” of B. subtilis genomic DNA pieces for sequencing uses inverse PCR. A known region is scanned for a set of appropriate restriction enzyme cleavage sites and inverse PCR is performed with a set of DNA primers determined from the outermost DNA sequence. The DNA fragments from the inverse PCR are directly used as template in the sequencing reaction. The newly derived sequences can be used to design new oligonucleotides. These new oligonucleotides are used to amplify DNA fragments with genomic DNA as template. The sequence determination on both strands of a DNA region is finished by applying a primer walking strategy on the genomic, PCR fragments. The benefit of multiple starting points in the primer walking results from the series of inverse PCR fragments with different sizes of new “cloned” DNA pieces. From the most external DNA sequence, a new round of inverse PCR is started. The whole inverse PCR strategy is based on the sequential use of conventional taq polymerase and the use of long range inverse PCR in those cases in which the taq polymerase failed to amplify DNA fragments. Nucleic acid sequencing is performed using standard technology. One method for nucleic acid sequencing involves the use of a Perkin-Elmer Applied Biosystems 373 DNA sequencer (Perkin-Elmer, Foster City, Calif.) according to manufacturer's instructions.

Nucleic acid sequences derived from genomic DNA may contain regulatory regions in addition to coding regions. Whatever the source, the isolated hydrolase gene should be molecularly cloned into a suitable vector for propagation of the gene.

In molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the hydrolase may be accomplished in a number of ways. For example, a B. subtilis hydrolase gene of the present invention or its specific RNA, or a fragment thereof, such as a probe or primer, may be isolated and labeled and then used in hybridization assays to detect a gram positive hydrolase gene. (Benton, W. and Davis, R., 1977, Science 196:180; Grunstein, M. and Hogness, D., 1975, Proc. Natl. Acad. Sci. USA 72:3961). Those DNA fragments sharing substantial sequence similarity to the probe will hybridize under stringent conditions.

Accordingly, the present invention provides a method for the detection of gram positive hydrolase polynucleotide homologues which comprises hybridizing part or all of a nucleic acid sequence of B. subtilis hydrolase with gram positive microorganism nucleic acid of either genomic or cDNA origin.

Also included within the scope of the present invention is the use of gram positive microorganism polynucleotide sequences that are capable of hybridizing to the nucleotide sequence of B. subtilis hydrolase under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol. 152, Academic Press, San Diego, Calif.) incorporated herein by reference, and confer a defined “stringency” as explained below.

“Maximum stringency” typically occurs at about Tm-5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. As will be understood by those of skill in the art, a maximum stringency hybridization can be used to identify or detect identical polynucleotide sequences while an intermediate or low stringency hybridization can be used to identify or detect polynucleotide sequence homologues.

The term “hybridization” as used herein shall include “the process by which a strand of nucleic acid joins with a complementary strand through base pairing” (Coombs, J., (1994), Dictionary of Biotechnology, Stockton Press, New York, N.Y.).

The process of amplification as carried out in polymerase chain reaction (PCR) technologies is described in Dieffenbach, C W and G S Dveksler, (PCR Primer, a Laboratory Manual, Cold Spring Harbor Press, Plainview, N.Y., 1995). A nucleic acid sequence of at least about 10 nucleotides and as many as about 60 nucleotides from B. subtilis hydrolase, preferably about 12 to 30 nucleotides, and more preferably about 20-25 nucleotides can be used as a probe or PCR primer.

The B. subtilis hydrolase amino acid sequences shown in the Sequences were identified via a BLAST search (Altschul, Stephen, Basic local alignment search tool, J. Mol. Biol., 215:403-410) of translated Bacillus subtilis genomic nucleic acid sequences. The conserved catalytic residues of the hydrolases are illustrated in Table II.

TABLE II

| hydrolase | nucleophile | acid | histidine |
|---|---|---|---|
| designation | residue | residue E/D | residue |
| YUXL¹ | GSY 518 | D599 | 631 |
| YTMA¹ | FSR 122 | D204 | 236 |
| YITV¹ | TSM 116 | D201 | 237 |
| YQKD¹ | ESM 160 | D249 | 278 |
| YCLE | HSG 95 | D232 | 261 |
| YTAP | MSM 169 | D244 | 281 |
| YDEN | HSL 71 | D137 | 161 |
| YBFK | LSL 129 | E244 | 273 |
| YFHM | HDW 102 | D237 | 265 |
| YDJP | WSM 94 | D217 | 246 |
| YVFQ² | HSV 96 | D219 | 247 |
| YVAM² | YSA 93 | D207 | 233 |
| YQJL | HSY 102 | | |
| SRFAD | HSM 86 | D 190 | H 210 |
| YCGS | ISA 68 | D 207 | H 235 |
| YTPA | HSM 88 | | |
| YBAC | HSW 115 | D 266 | H 296 |
| YUII | HSL 188 | | |
| YODD | YSN 100 | | |
| YJCH | DSL 129 | | |
| YODH | RSY 172 | | |

¹hydrolases appear to have overall amino acid similarity to one another
²hydrolases appear to have overall amino acid similarity to one another

II. Expression Systems

The present invention provides host cells, expression methods and systems for the enhanced production and secretion of gram positive microorganism hydrolases. In one embodiment of the present invention, the host cell is a gram negative host cell and in another embodiment, the host cell is a gram positive host cell. The host cell may also be a fungal or mammalian host cell. In one embodiment of the present invention, a gram positive or gram negative microorganism is genetically engineered to produce and/or overproduce a hydrolase of the present invention.

III. Production of Hydrolase

For production of hydrolase in a host cell, an expression vector comprising at least one copy of nucleic acid encoding a gram positive microorganism hydrolase, and preferably comprising multiple copies, is transformed into the host cell under conditions suitable for expression of the hydrolase. In accordance with the present invention, polynucleotides which encode a gram positive microorganism hydrolase, or fragments thereof, or fusion proteins or polynucleotide homologue sequences that encode amino acid variants of B. subtilis hydrolase, may be used to generate recombinant DNA molecules that direct their expression in host cells. In a preferred embodiment, the gram positive host cell belongs to the genus Bacillus. In a further preferred embodiment, the gram positive host cell is B. subtilis.

As will be understood by those of skill in the art, it may be advantageous to produce polynucleotide sequences possessing non-naturally occurring codons. Codons preferred by a particular gram positive host cell (Murray, E. et al., (1989), Nuc. Acids Res. 17:477-508) can be selected, for example, to increase the rate of expression or to produce recombinant RNA transcripts having desirable properties, such as a longer half-life, than transcripts produced from a naturally occurring sequence.

Altered hydrolase polynucleotide sequences which may be used in accordance with the invention include deletions, insertions or substitutions of different nucleotide residues resulting in a polynucleotide that encodes the same or a functionally equivalent hydrolase homologue, respectively. As used herein a “deletion” is defined as a change in the nucleotide sequence of the hydrolase resulting in the absence of one or more amino acid residues.

As used herein, an “insertion” or “addition” is that change in the nucleotide sequence which results in the addition of one or more amino acid residues as compared to the naturally occurring hydrolase.

As used herein, “substitution” results from the replacement of one or more nucleotides or amino acids by different nucleotides or amino acids, respectively. The change(s) in the nucleotides(s) can either result in a change in the amino acid sequence or not.

The encoded protein may also show deletions, insertions or substitutions of amino acid residues which produce a silent change and result in a functionally equivalent hydrolase variant. Deliberate amino acid substitutions may be made on the basis of similarity in polarity, charge, solubility, hydrophobicity, hydrophilicity, and/or the amphipathic nature of the residues as long as the variant retains its proteolytic ability. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine, phenylalanine, and tyrosine.

The hydrolase polynucleotides of the present invention may be engineered in order to modify the cloning, processing and/or expression of the gene product. For example, mutations may be introduced using techniques which are well known in the art, i.e., site-directed mutagenesis to insert new restriction sites, to alter glycosylation patterns or to change codon preference, for example.

In one embodiment of the present invention, a gram positive microorganism hydrolase polynucleotide may be ligated to a heterologous sequence to encode a fusion protein. A fusion protein may also be engineered to contain a cleavage site located between the hydrolase nucleotide sequence and the heterologous protein sequence, so that the hydrolase may be cleaved and purified away from the heterologous moiety.

IV. Vector Sequences

Expression vectors used in expressing the hydrolases of the present invention in gramn positive microorganisms comprise at least one promoter associated with the hydrolase, which promoter is functional in the host cell. In one embodiment of the present invention, the promoter is the wild-type promoter for the selected hydrolase and in another embodiment of the present invention, the promoter is heterologous to the hydrolase, but still functional in the host cell. In one preferred embodiment of the present invention, nucleic acid encoding the hydrolase is stably integrated into the microorganism genome.

In a preferred embodiment, the expression vector contains a multiple cloning site cassette which preferably comprises at least one restriction endonuclease site unique to the vector, to facilitate ease of nucleic acid manipulation. In a preferred embodiment, the vector also comprises one or more selectable markers. As used herein, the term “selectable marker” refers to a gene capable of expression in the gram positive host which allows for ease of selection of those hosts containing the vector. Examples of such selectable markers include but are not limited to antibiotics, such as, erythromycin, actinomycin, chloramphenicol and tetracycline.

V. Transformation

A variety of host cells can be used for the production Bacillus subtilis hydrolase or hydrolase homologues including bacterial, fungal, mammalian and insects cells. General transformation procedures are taught in Current Protocols In Molecular Biology, (Vol. 1, edited by Ausubel et al., John Wiley & Sons, Inc., 1987, Chapter 9) and include calcium phosphate to methods, transformation using DEAE-Dextran and electroporation. Plant transformation methods are taught in Rodriquez (WO 95/14099, published 26 May 1995).

In a preferred embodiment, the host cell is a gram positive microorganism and in another preferred embodiment, the host cell is Bacillus. In one embodiment of the present invention, nucleic acid encoding one or more hydrolase(s) of the present invention is introduced into a host cell via an expression vector capable of replicating within the Bacillus host cell. Suitable replicating plasmids for Bacillus are described in Molecular Biological Methods for Bacillus, Ed. Harwood and Cutting, John Wiley & Sons, 1990, hereby expressly incorporated by reference; see chapter 3 on plasmids. Suitable replicating plasmids for B. subtilis are listed on page 92.

In another embodiment, nucleic acid encoding a hydrolase(s) of the present invention is stably integrated into the microorganism genome. Preferred host cells are gram positive host cells. Another preferred host is Bacillus. Another preferred host is Bacillus subtilis. Several strategies have been described in the literature for the direct cloning of DNA in Bacillus. Plasmid marker rescue transformation involves the uptake of a donor plasmid by competent cells carrying a partially homologous resident plasmid (Contente et al., Plasmid, 2:555-571 (1979); Haima et al., Mol. Gen. Genet., 223:185-191 (1990); Weinrauch et al., J. Bacteriol., 154(3):1077-1087 (1983); and Weinrauch et al., J. Bacteriol., 169(3):1205-1211 (1987)). The incoming donor plasmid recombines with the homologous region of the resident “helper” plasmid in a process that mimics chromosomal transformation.

Transformation by protoplast transformation is described for B. subtilis in Chang and Cohen, (1979), Mol. Gen. Genet., 168:111-115; for B. megaterium in Vorobjeva et al., (1980), FEMS Microbiol. Letters, 7:261-263; for B. amyloliquefaciens in Smith et al., (1986), Appl. and Env. Microbiol., 51:634; for B. thuringiensis in Fisher et al., (1981), Arch. Microbiol., 139:213-217; for B. sphaericus in McDonald, (1984), J. Gen. Microbiol., 130:203; and B. larvae in Bakhiet et al., (1985, Appl. Environ. Microbiol. 49:577). Mann et al., (1986, Current Microbiol., 13:131-135) report on transformation of Bacillus protoplasts and Holubova, (1985), Folia Microbiol., 30:97) disclose methods for introducing DNA into protoplasts using DNA containing liposomes.

VI. Identification of Transformants

Whether a host cell has been transformed with a mutated or a naturally occurring gene encoding a gram positive hydrolase detection of the presence/absence of marker gene expression can suggest whether the gene of interest is present. However, its expression should be confirmed. For example, if the nucleic acid encoding a hydrolase is inserted within a marker: gene sequence, recombinant cells containing the insert can be identified by the absence of marker gene function. Alternatively, a marker gene can be placed in tandem with nucleic acid encoding the hydrolase under the control of a single promoter. Expression of the marker gene in response to induction or selection usually indicates expression of the hydrolase as well.

Alternatively, host cells which contain the coding sequence for a hydrolase and express the protein may be identified by a variety of procedures known to those of skill in the art. These is procedures include, but are not limited to, DNA-DNA or DNA-RNA hybridization and protein bioassay or immunoassay techniques which include membrane-based, solution-based, or chip-based technologies for the detection and/or quantification of the nucleic acid or protein.

The presence of the hydrolase polynucleotide sequence can be detected by DNA-DNA or, DNA-RNA hybridization or amplification using probes, portions or fragments of B. subtilis hydrolase.

VII. Assay of Activity

There are various assays known to those of skill in the art for detecting and measuring hydrolytic activity. There are assays based upon the release of acid-soluble peptides from casein or hemoglobin measured as absorbance at 280 nm or colorimetrically using the Folin method (Bergmeyer, et al., 1984, Methods of Enzymatic Analysis, Vol. 5, Peptidases, Proteinases and their Inhibitors, Verlag Chemie, Weinheim). Other assays involve the solubilization of chromogenic substrates (Ward, 1983, Proteinases, in Microbial Enzymes and Biotechnology, (W. M. Fogarty, ed.), Applied Science, London, pp. 251-317). Other assays for specific hydrolases, such as esterases, lipases, peroxidases, are known by those of skill in the art.

VIII. Secretion of Recombinant Proteins

Means for determining the levels of secretion of a heterologous or homologous protein in a gram positive host cell and detecting secreted proteins include using either polyclonal or monoclonal antibodies specific for the protein. Examples include enzyme-linked immunosorbent assay (ELISA), radioimmunoassay (RIA) and fluorescent activated cell sorting (FACS). These and other assays are described, among other places, in Hampton, R. et al., (1990, Serological Methods, a Laboratory Manual, APS Press, St. Paul Minn.) and Maddox, D E et al., (1983, J. Exp. Med., 158:1211).

A wide variety of labels and conjugation techniques are known by those skilled in the art and can be used in various nucleic and amino acid assays. Means for producing labeled hybridization or PCR probes for detecting specific polynucleotide sequences include oligolabeling, nick translation, end-labeling or PCR amplification using a labeled nucleotide. Alternatively, the nucleotide sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides.

A number of companies such as Pharmacia Biotech (Piscataway N.J.), Promega (Madison Wis.), and US Biochemical Corp. (Cleveland Ohio) supply commercial kits and protocols for these is procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced as shown in U.S. Pat. No. 4,816,567 and incorporated herein by reference.

IX. Purification of Proteins

Gram positive host cells transformed with polynucleotide sequences encoding hydrolases may be cultured under conditions suitable for the expression and recovery of the hydrolase from cell culture. Other recombinant constructions may join the hydrolase sequences to a nucleotide sequence encoding a polypeptide domain which will facilitate purification of soluble proteins (Kroll, D J. et al., (1993), DNA Cell Biol., 12:441-53).

Such purification facilitating domains include, but are not limited to, metal chelating peptides such as histidine-tryptbphan modules that allow purification on immobilized metals (Porath, J., (1992), Protein Expr. Purif. 3:263-281), protein A domains that allow purification on immobilized immunoglobulin, and the domain utilized in the FLAGS extension/affinity purification system (Immunex Corp, Seattle Wash.). The inclusion of a cleavable linker sequence such as Factor XA or enterokinase (Invitrogen, San Diego Calif.) between the purification domain and the heterologous protein can be used to facilitate purification.

X. Uses of the Present Invention

Hydrolase and Genetically Engineered Host Cells

The present invention provides genetically engineered host cells comprising nucleic acid encoding hydrolases of the present invention. The host cell may contain other modifications, e.g., protease deletions, such as deletions of the mature subtilisn protease and/or mature neutral protease disclosed in U.S. Pat. No. 5,264,366 or other modifications to enhance expression.

In a preferred embodiment, the host cell is a Bacillus. In a further preferred embodiment, the host cell is a Bacillus subtilis.

In a preferred embodiment, the host cell is grown under large scale fermentation conditions. In another preferred embodiment, the hydrolase is isolated and/or purified and used in the textile industry, the feed industry and in cleaning compositions such as detergents.

As noted, hydrolase can be useful in formulating various cleaning compositions. A number of known compounds are suitable surfactants useful in compositions comprising the hydrolase of the invention. These include nonionic, anionic, cationic, anionic or zwitterionic detergents, as disclosed in U.S. Pat. No. 4,404,128 and U.S. Pat. No. 4,261,868. A suitable detergent formulation is that described in Example 7 of U.S. Pat. No. 5,204,015. The art is familiar with the different formulations which can be used as cleaning compositions. In addition, hydrolase can be used, for example, in bar or liquid soap applications, dishcare formulations, contact lens cleaning solutions or products, peptide hydrolysis, waste treatment, textile applications, as fusion-cleavage enzymes in protein production, etc. hydrolase may comprise enhanced performance in a detergent composition (as compared to another detergent protease). As used herein, enhanced performance in a detergent is defined as increasing cleaning of certain enzyme sensitive stains such as grass or blood, as determined by usual evaluation after a standard wash cycle.

Hydrolases of the present invention can be formulated into known powdered and liquid detergents having pH between 6.5 and 12.0 at levels of about 0.01 to about 5% (preferably 0.1% to 0.5%) by weight. These detergent cleaning compositions can also include other enzymes such as known proteases, amylases, cellulases, lipases or endoglycosidases, as well as builders and stabilizers.

The addition of hydrolase to conventional cleaning compositions does not create any special use limitation. In other words, any temperature and pH suitable for the detergent is also suitable for the present compositions as long as the pH is within the above range, and the temperature is below the described hydrolase denaturing temperature. In addition, hydrolase can be used in a cleaning composition without detergents, again either alone or in combination with builders and stabilizers.

Hydrolases can be included in animal feed such as part of animal feed additives as described in, for example, U.S. Pat. No. 5,612,055; U.S. Pat. No. 5,314,692; and U.S. Pat. No. 5,147,642.

One aspect of the invention is a composition for the treatment of a textile that comprises a hydrolase. The composition can be used to treat for example silk or wool as described in publications such as RD 216,034; EP 134,267; U.S. Pat. No. 4,533,359; and EP 344,259.

Hydrolase Polynucleotides

A B. subtlis hydrolase polynucleotide, or any part thereof, provides the basis for detecting the presence of gram positive microorganism hydrolase polynucleotide homologues through hybridization techniques and PCR technology.

Accordingly, one aspect of the present invention is to provide for nucleic acid hybridization and PCR probes which can be used to detect polynucleotide sequences, including genomic and cDNA sequences, encoding gram positive hydrolase or portions thereof.

The manner and method of carrying out the present invention may be more fully is understood by those of skill in the art by reference to the following examples, which examples are not intended in any manner to limit the scope of the present invention or of the claims directed thereto

### EXAMPLE I

### Preparation of a Genomic Library

The following example illustrates the preparation of a Bacillus genomic library.

Genomic DNA from Bacillus cells is prepared as taught in Current Protocols In Molecular Biology. Vol. 1, edited by Ausubel et al., John Wiley & Sons, Inc., 1987, Chapter 2. 4.1. Generally, Bacillus cells from a saturated liquid culture are lysed and the proteins removed by digestion with proteinase K. Cell wall debris, polysaccharides, and remaining proteins are removed by selective precipitation with CTAB, and high molecular weight genomic DNA is recovered from the resulting supernatant by isopropanol precipitation. If exceptionally clean genomic DNA is desired, an additional step of purifying the Bacillus genomic DNA on a cesium chloride gradient is added.

After obtaining purified genomic DNA, the DNA is subjected to Sau3A digestion. Sau3A recognizes the 4 base pair site GATC and generates fragments compatible with several convenient phage lambda and cosmid vectors. The DNA is subjected to partial digestion to increase the chance of obtaining random fragments.

The partially digested Bacillus genomic DNA is subjected to size fractionation on a 1% agarose gel prior to cloning into a vector. Alternatively, size fractionation on a sucrose gradient can be used. The genomic DNA obtained from the size fractionation step is purified away from the agarose and ligated into a cloning vector appropriate for use in a host cell and transformed into the host cell.

### EXAMPLE II

### Detection of Gram Positive Microorganism Hydrolase

The following example describes the detection of gram positive microorganism hydrolase

DNA derived from a gram positive microorganism is prepared according to the methods disclosed in Current Protocols in Molecular Biology, Chap. 2 or 3. The nucleic acid is subjected to hybridization and/or PCR amplification with a probe or primer derived from hydrolase

The nucleic acid probe is labeled by combining 50 pmol of the nucleic acid and 250 mCi of [gamma ³²P] adenosine triphosphate (Amersham, Chicago Ill.) and T4 polynucleotide kinase (DuPont NEN®, Boston Mass.). The labeled probe is purified with Sephadex G-25 super fine resin column (Pharmacia). A portion containing 10⁷ counts per minute of each is used in a typical membrane based hybridization analysis of nucleic acid sample of either genomic or cDNA origin.

The DNA sample which has been subjected to restriction endonuclease digestion is fractionated on a 0.7 percent agarose gel and transferred to nylon membranes (Nytran Plus, Schleicher & Schuell, Durham N.H.). Hybridization is carried out for 16 hours at 40 degrees C. To remove nonspecific signals, blots are sequentially washed at room temperature under increasingly stringent conditions up to 0.1×saline sodium citrate and 0.5% sodium dodecyl sulfate. The blots are exposed to film for several hours, the film developed and hybridization patterns are compared visually to detect polynucleotide homologues of B. subtilis hydrolase. The homologues are subjected to confirmatory nucleic acid sequencing. Methods for nucleic acid sequencing are well known in the art. Conventional enzymatic methods employ DNA polymerase Klenow fragment, SEQUENASE® (US Biochemical Corp, Cleveland, Ohio) or Taq polymerase to extend DNA chains from an oligonucleotide primer annealed to the DNA template of interest.

Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

1. A gram positive microorganism comprising an isolated nucleic acid encoding a hydrolase selected from the group consisting of YUXL, YTMA, YITV, YQKD, YCLE, YTAP, YDEN, YBFK, YFHM, YDJP, YVFQ, YVAM, YQJL, SRFAD, YCGS, YTPA, YBAC, YUII, YCLE, YODD, YJCH, YODH.

2. The gram positive microorganism according to claim 1 that is a member of the family Bacillus.

3. The microorganism according to claim 2 wherein the Bacillus includes B. subtilis, B. licheniformis, B. lentus, B. brevis, B. stearothermophilus, B. alkalophilus, B. amyloliquefaciens, B. coagulans, B. circulans, B. lautus and B. thuringiensis.

4. The microorganism of claim 3 wherein said microorganism comprises a deletion in a naturally occurring protease.

5. A cleaning composition comprising a hydrolase selected from the group consisting of YUXL, YTMA, YITV, YQKD, YCLE, YTAP, YDEN, YBFK, YFHM, YDJP, YVFQ, YVAM, YQJL, SRFAD, YCGS, YTPA, YBAC, YUII, YCLE, YODD, YJCH, YODH.

6. The cleaning composition according to claim 5 wherein the hydrolase has at least 80% homology with the naturally occurring hydrolase.

7. An animal feed comprising a hydrolase selected from the group consisting of YUXL, YTMA, YITV, YQKD, YCLE, YTAP, YDEN, YBFK, YFHM, YDJP, YVFQ, YVAM, YQJL, SRFAD, YCGS, YTPA, YBAC, YUII, YCLE, YODD, YJCH, YODH.

8. The animal feed of claim 7 wherein the hydrolase has at least 80% homology with the naturally occurring hydrolase.

9. A composition for the treatment of a textile comprising a hydrolase selected from the group consisting of YUXL, YTMA, YITV. YQKD, YCLE, YTAP, YDEN, YBFK. YFHM, YDJP, YVFQ, YVAM, YQJL, SRFAD, YCGS, YTPA, YBAC, YUII, YCLE, YODD, YJCH, YODH.

10. The composition of claim 9 wherein the hydrolase has at least 80% homology to the naturally occurring hydrolase.

11. An expression vector comprising a nucleic acid encoding a gram positive hydrolase selected from the group consisting of YUXL, YTMA, YITV, YQKD, YCLE, YTAP, YDEN, YBFK, YFHM, YDJP, YVFQ, YVAM, YQJL, SRFAD, YCGS, YTPA, YBAC, YUII, YCLE, YODD, YJCH, YODH.

12. A host cell comprising an expression vector according to claim 11.

13. A method for detecting a gram positive microorganism hydrolase, comprising the steps of
(a) hybridizing a gram positive microorganism nucleic acid to a probe, wherein the probe comprises part or all of the naturally occurring nucleic acid sequence encoding a hydrolase selected from the group consisting of YUXL, YTMA, YITV, YQKD, YCLE, YTAP, YDEN, YBFK, YFHM, YDJP, YVFQ, YVAM, YQJL, SRFAD, YCGS, YTPA, YBAC, YUII, YCLE, YODD, YJCH, YODH; and
(b) isolating the gram positive nucleic acid which hybridizes to said probe.

14. The method of claim 13, wherein the hybridization takes place under low stringency conditions.


SEQUENCE LISTING (21 sequences)

SEQ ID NO: 1 - protein, 657 aa, Bacillus
MKKLITADDITAIVSVTDPQYAPDGTRAAYVKSQVNQEKDSYTSNIWIYETKTGGSVPWTHGEKRSTDPRWSPDGRTLAFISDREGDAAQLYIMSTEGGEARKLTDIPYGVSKPLWSPDGESILVTISLGEGESIDDREKTEQDSYEPVEVQGLSYKRDGKGLTRGAYAQLVLVSVKSGEMKELTSHKADHGDPAFSPDGKWLVFSANLTETDDASKPHDVYIMSLESGDLKQVTPHRGSFGSSSFSPDGRYLALLGNEKEYKNATLSKAWLYDIEQGRLTCLTEMLDVHLADALIGDSLIGGAEQRPIWTKDSQGFYVIGTDQGSTGIYYISIEGLVYPIRLEKEYINSFSLSPDEQHFIASVTKPDRPSELYSIPLGQEEKQLTGANDKFVREHTISIPEEIQYATEDGVMVNGWLMRPAQMEGETTYPLILNIHGGPHMMYGHTYFHEFQVLAAKGYAVVYINPRGSHGYGQEFVNAVRGDYGGKDYDDVMQAVDEAIKRDPHIDPKRLGVTGGSYGGFMTNWIVGQTNRFKAAVTQRSISNWISFHGVSDIGYFFTDWQLEHDMFEDTEKLWDRSPLKYAANVETPLLILHGERDDRCPIEQAEQLFIALKKMGKETKLVRFPNASHNLSRTGHPRQRIKRLNYISSWFDQHL

SEQ ID NO: 2 - protein, 256 aa, Bacillus
MIVEKRRFPSPSQHVRLYTICYLSNGLRVKGLLAEPAEPGQYDGFLYLRGGIKSVGMVRPGRIIQFASQGFVVFAPFYRGNQGGEGNEDFAGEDREDAFSAFRLLQQHPNVKKDRIHIFGFSRGGIMGMLTAIEMGGQAASFVSWGGVSDMILTYEERQDLRRMMKRVIGGTPKKVPEEYQWRTPFDQVNKIQAPVLLIHGEKDQNVSIQHSYLLEEKLKQLHKPVETWYYSTFTHYFPPKENRRIVRQLTQWMKN

SEQ ID NO: 3 - protein, 255 aa, Bacillus
MIQIENQTVSGIPFLHIVKEENRHRAVPLVIFIHGFTSAKEHNLHIAYLLAEKGFRAVLPEALHHGERGEEMAVEELAGHFWDIVLNEIEEIGVLKNHFEKEGLIDGGRIGLAGTSMGGITTLGALTAYDWIKAGVSLMGSPNYVELFQQQIDHIQSQGIEIDVPEEKVQQLMKRLELRDLSLQPEKLQQRPLLFWHGAKDKVVPYAPTRKFYDTIKSHYSEQPERLQFIGDENADHKVPRAAVLKTIEWFETYL

SEQ ID NO: 4 - protein, 300 aa, Bacillus
MKKILLAIGALVTAVIAIGIVFSHMILFIKKKTDEDIIKRETDNGHDVFESFEQMEKTAFVIPSAYGYDIKGYHVAPHDTPNTIIICHGVTMNVLNSLKYMHLFLDLGWNVLIYDHRRHGQSGGKTTSYGFYEKDDLNKVVSLLKNKTNHRGLIGIHGESMGAVTALLYAGAHCSDGADFYIADCPFACFDEQLAYRLRAEYRLPSWPLLPIADFFLKLRGGYRAREVSPLAVIDKIEKPVLFIHSKDDDYIPVSSTERLYEKKRGPKALYIAENGEHAMSYTKNRHTYRKTVQEFLDNM

SEQ ID NO: 5 - protein, 281 aa, Bacillus
MERAGICHSDGFDLAYRIEGEGAPILVIGSAAYYPRLFSSDIKQKYQWVFVDHRGFAKPKRELRAEDSRLDAVLADIERMRTFLQLEDVTTLGHSGHAFMALEYARTYPKQVRKVALFNTAPDNSEERQRKSESFFMETASLERKKRFEKDIENLPQDIDKDPERRFVHMCIRAEAKSFYQERPHAAALWDGVFTNMPIIDELWGNTFARIDLLQRLADVRMPVYIGLGRYDYLVAPVSLWDAVDGLYPHVDKVIFEKSGHQPMLEEPEAFDQSFRKWLDQ

SEQ ID NO: 6 - protein, 298 aa, Bacillus
MRAERRKQLFRLLGDLPDRRPISVETLRIEEREENIVETLLLDLNGHEKAPAYFVKPKKTEGPCPAVLFQHSHGGQYDRGKSELIEGADYLKTPSFSDELTSLGYGVLAIDHWGFGDRRGKAESEIFKEMLLTGKVMWGMMIYDSLSALDYMQSRSDVQPDRIGTIGMSMGGLMAWWTAALDDRIKVCVDLCSQVDHHVLIKTQNLDRHGFYYYVPSLAKHFSASEIQSLIAPRPHLSLVGVHDRLTPAEGVDKIEKELTAVYAGQGAADCYRVVRSASGHFETAVIRHEAVRFLQKW

SEQ ID NO: 7 - protein, 190 aa, Bacillus
MTKQVYIIHGYRASSTNHWFPWLKKRLLADGVQADILNMPNPLQPRLEDWLDTLSLYQHTLHENTYLVAHSLGCPAILRFLEHLQLRKQLGGIILVSGFAKSLPTLQMLDEFTQGSFDHQKIIESAKHRAVIASKDDQIVPFSFSKDLAQQIDAALYEVQHGGHFLEDEGFTSLPIVYDVLTSYFSKETR

SEQ ID NO: 8 - protein, 296 aa, Bacillus
MIQDSMQFAAVESGLRFYQAYDQSLSLWPIESEAFYVSTRFGKTHIIASGPKDAPSLILLHGGLFSSAMWYPNIAAWSSQFRTYAVDIIGDKNKSIPSAAMETRADFAEWMKDVFDSLGLETAHLAGLSLGGSHIVNFLLRAPERVERAVVISPAEAFISFHPDVYKYAAELTGARGAESYIKWITGDSYDLHPLLQRQIVAGVEWQDEQRSLKPTENGFPYVFTDQELKSIQVPVLLMFGEHEAMYHQQMAFERASVLVPGIQAEIVKNAGHLLSLEQPEYVNQRVLSFLCGGIK

SEQ ID NO: 9 - protein, 286 aa, Bacillus
MDGVKCQFVNTNGITLHVAAAGREDGPLIVLLHGFPEFWYGWKNQIKPLVDAGYRVIAPDQRGYNLSDKPEGIDSYRIDTLRDDIIGLITQFTDEKAIVIGHDWGGAVAWHLASTRPEYLEKLIAINIPHPHVMKTVTPLYPPQWLKSSYIAYFQLPDIPEASLRENDYDTLDKAIGLSDRPALFTSEDVSRYKEAWKQPGALTAMLNWYRALRKGSLAEKPSYETVPYRMIWGMEDRFLSRKLAKETERHCPNGHLIFVDEASHWINHEKPAIVNQLILEYLKNQ

SEQ ID NO: 10 - protein, 271 aa, Bacillus
MPYIILEDQTRLYYETHGSGTPILFIHGVLMSGQFFHKQFSVLSANYQCIRLDLRGHGESDKVLHGHTISQYARDIREFLNAMELDHVVLAGWSMGAFVVWDYLNQFGNDNIQAAVIIDQSASDYQWEGWEHGPFDFDGLKTAMHAIQTDPLPFYESFIQNMFAEPPAETETEWMLAEILKQPAAISSTILFNQTAADYRGTLQNINVPALLCFGEDKKFFSTAAGEHLRSNIPNATLVTFPKSSHCPFLEEPDAFNSTLLSFLDGVIGKS

SEQ ID NO: 11 - protein, 269 aa, Bacillus
MNEAILSRNHVKVKGSGKASIMFAPGFGCDQSVWNAVAPAFEEDHRVILFDYVGSGHSDLRAYDLNRYQTLDGYAQDVLDVCEALDLKETVFVGHSVGALIGMLASIRRPELFSHLVMVGPSPCYLNDPPEYYGGFEEEQLLGLLEMMEKNYIGWATVFAATVLNQPDRPEIKEELESRFCSTDPVIARQFAKAAFFSDHREDLSKVTVPSLILQCADDIIAPATVGKYMHQHLPYSSLKQMEARGHCPHMSHPDETIQLIGDYLKAHV

SEQ ID NO: 12 - protein, 256 aa, Bacillus
MPLISIDSRKHLFYEEYGQGIPIIFIHPPGMGRKVFYYQRLLSKHFRVIFPDLSGHGDSDHIDQPASISYYANEIAQFMDALHIDKAVLFGYSAGGLIAQHIGFTRPDKVSHLILSGAYPAVHNVIGQKLHKLGMYLLEKNPGLLMKILAGSHTKDRQLRSILTDHMKKADQAHWHQYYLDSLGYNCIEQLPRLEMPMLFMYGGLRDWTFTNAGYYRRSCRHAEFFRLEYQGHQLPTKQWKTCNELVTGFVLTHHS

SEQ ID NO: 13 - protein, 253 aa, Bacillus
MKSAWMEKTYTIDGCAFHTQHRKGSSGVTIVFEAGYGTSSETWKPLMADIDDEFGIFTYDRAGIGKSGQSRAKRTADQQVKELESLLKAADVKPPYLAVSHSYGAVITGLWACKNKHDIIGMVLLDPALGDCASFTFIPEEMHKSHTRKMMLEGTHAEFSKSLQELKKRQVHLGNMPLLVLSSGERTEKFAAEQEWQNLHSSILSLSNQSGWIQAKNSSHNIHHDEPHIVHLAIYDVWCAACQQAAPLYQAVN

SEQ ID NO: 14 - protein, 242 aa, Bacillus
MSQLFKSFDASEKTQLICFPFAGGYSASFRPLHAFLQGECEMLAAEPPGHGTNQTSAIEDLEELTDLYKQELNLRPDRPFVLFGHSMGGMITFRLAQKLEREGIFPQAVIISAIQPPHIQRKKVSHLPDDQFLDHIIQLGGMPAELVENKEVMSFFLPSFRSDYRALEQFELYDLAQIQSPVHVFNGLDDKKCIRDAEGWKKWAKDITFHQFDGGHMFLLSQTEEVAERIFAILNQHPIIQP

SEQ ID NO: 15 - protein, 256 aa, Bacillus
MHGGHSNCYEEFGYTALIEQGYSIITPSRPGYGRTSKEIGKSLANACRFYVKLLDHLQIESVHVIAISAGGPSGICFASHYPERVNTLTLQSAVTKEWLTPKDTEYKLGEILFRPPVEKWIWKLISSLNNAFPRLMFRAMSPQFSTLPFQRIKSLMNEKDIEAFRKMNSRQRSGEGFLIDLSQTAAVSLKDLQAIICPVLIMQSVYDGLVDLSHAHHAKEHIRGAVLCLLHSWGHLIWLGKEAAETGSILLGFLES

SEQ ID NO: 16 - protein, 318 aa, Bacillus
MIPEKKSIAIMKELSIGNTKQMLMINGVDVKNPLLLFLHGGPGTPQIGYVRHYQKELEQYFTVVHWDQRGSGLSYSKRISHHSMTINHFIKDTIQVTQWLLAHFSKSKLYLAGHSWGSILALHVLQQRPDLFYTYYGISQVVNPQDEESTAYQHIREISESKKASILSFLTRFIGAPPWKQDIQHLIYRFCVELTRGGFTHRHRQSLAVLFQMLTGNEYGVRNMHSFLNGLRFSKKHLTDELYRFNAFTSVPSIKVPCVFISGKHDLIVPAEISKQYYQELEAPEKRWFQFENSAHTPHIEEPSLFANTLSRHARNHL

SEQ ID NO: 17 - protein, 314 aa, Bacillus
MRCLVDSENHYHTLRFSLRRGMSYCMKEQTTDRTNGGTSNAFTIPGTEVRMMSSRNENRTYHIFISKPSTPPPPAGYPVIYLLDANSVFGTMTEAVRIQGRRPEKTGVIPAVIVGIGYETAEPFSSARHRDFTMPTAQSKLPERPDGREWPEHGGAEGFFRFIEEDLKPEIERDYQIDKKRQTIFGHSLGGLFVLQVLLTKPDAFQTYIAGSPSIHWNKPFILKKTDHFVSLTKKNNQPINILLAAGELEQHHKSRMNDNARELYERLAVLSEQGIRAEFCEFSGEGHISVLPVLVSRALRFALHPDGPHLSMG

SEQ ID NO: 18 - protein, 200 aa, Bacillus
MKHIYEKGTSDNVLLLLHGTGGNEHDLLSLGRFIDPDAHLLGVRGSVLENGMPRFFKRLSEGVFDEKDLVVRTRELKDFIDEAAETHQFNRGRVIAVGYSNGANIAASLLFHYKDVLKGAILHHPMVPIRGIELPDMAGLPVFIGAGKYDPLCTKEESEELYRYLRDSGASASVYWQDGGHQLTQHEAEQAREWYKEAIV

SEQ ID NO: 19 - protein, 240 aa, Bacillus
MAPKNGTVQEKKFFSKELNEEMTLLVYLPSNYSPLYKYHVIIAQDGHDYFRLGRIGRQVEELLSKREIDRSIIIGVPYKDVKERRNTYHPEGSKFSAYKRFIAHELVPFADDEYPTYQIGYGRTLIGDSLGATVSLMTALDYPNMFGNIIMQSPYVDKHVLEAVKQSDDIKHLSIYHQIGTKETDVHTTDGNILDFTEPNRELKQLLEKKLSDYDFEPFDGDHKWTYWQPLITPALKKML

SEQ ID NO: 20 - protein, 233 aa, Bacillus
MSRYLEMLSLFGVAGAHPGGLAFSKAVLQKAAPSPDQPILDAGCGTGQTAAYLGHLLYPVTVVDKDPIMLEKAKKRFANEGLAIPAYQAELEHLPFSSESFSCVLSESVLSFSRLTSSLQEISRVLKPSGMLIGIEAALKKPMPPAEKKQMMDFYGFTCLHEESEWHKLLRSYGFQKTEAMSLLPEDMEFEPTTEMDLSQTIDPIYYDTLQTHYQLMQLYSEYMGHCIFIAYK

SEQ ID NO: 21 - protein, 259 aa, Bacillus
MWTWKADRPVAVIVIIHGASEYHGRYKWLIEMWRSSGYHVVMGDLPGQGTTTRARGHIRSFQEYIDEVDAWIDKARTFDLPVFLLGHSMGGLVAIEWVKQQRNPRITGIILSSPCLGLQIKVNKALDLASKGLNVIAPSLKVDSGLSIDMATRNEDVIEADQNDSLYVRKVSVRWYRELLKTIESAMVPTEAFLKVPLLVMQAGDDKLVDKTMVIKWFNGVASHNKAYREWEGLYHEIFNEPEREDVFKAARAFTDQYI